(19) United States
(12) Patent Application Publication
(10) Pub. No.: US 2026/0115435 A1
(43) Pub. Date: Apr. 30, 2026

(54) PULMONARY VEIN SHUNTING

(71) Applicant: Edwards Lifesciences Corporation, Irvine, CA (US)

(72) Inventors: Hao Shang, Irvine, CA (US); Zheng Lin, Fountain Valley, CA (US)

(21) Appl. No.: 19/431,862

(22) Filed: Dec. 23, 2025

Related U.S. Application Data
(63) Continuation of application No. 17/588,856, filed on Jan. 31, 2022, now Pat. No. 12,527,942.
(60) Provisional application No. 63/144,218, filed on Feb. 1, 2021.

(51) Int. Cl. A61M 27/00; A61B 18/00; A61B 18/14; A61F 2/86; A61M 29/00

(52) U.S. Cl. CPC A61M 27/002; A61B 18/14; A61F 2/86; A61M 29/00; A61B 2018/00577; A61B 2018/00601; A61B 2018/1412; A61M 2210/125

(57) ABSTRACT

Methods for reducing cardiac pressure involve advancing a surgical instrument into a left atrium of a heart and into a pulmonary vein fluidly coupled to the left atrium. A first opening is formed in a wall portion of the pulmonary vein and a second opening is formed in a wall portion of a pulmonary artery. A shunt implant is deployed through the first and second openings such that the shunt implant extends between the pulmonary vein and the pulmonary artery and defines a shunt flow path therebetween. Blood is shunted from the pulmonary vein into the pulmonary artery through the shunt flow path. In some implementations, the pulmonary vein and the pulmonary artery are drawn toward one another prior to deployment of the shunt implant, and the shunt implant clamps tissue of the pulmonary vein and tissue of the pulmonary artery together to establish a fluid-tight shunt pathway.

### RELATED APPLICATIONS

This is a continuation of U.S. patent application Ser. No. 17/588,856, filed Jan. 31, 2022, which claims the benefit of U.S. Provisional Patent Application Ser. No. 63/144,218, filed Feb. 1, 2021, the complete disclosures of each of which are hereby incorporated herein by reference in their entireties.

### BACKGROUND

### Field

The present disclosure generally relates to the field of medical devices and procedures.

### Description of Related Art

Certain physiological parameters associated with chambers of the heart, such as fluid pressure, can have an impact on patient health prospects. In particular, high cardiac fluid pressure can lead to heart failure and/or other complications in some patients. Therefore, reduction of pressure in certain chambers and vessels of the heart can improve patient health in some cases.

### SUMMARY

Described herein are devices, methods, and systems that facilitate shunting of blood from the left side of the heart to the right side of the heart via one or more of the pulmonary veins. Devices associated with the various embodiments of the present disclosure can include shunt structures configured to retain blood flow pathways through pulmonary vein walls. Methods associated with the various embodiments of the present disclosure can involve forming shunt openings in pulmonary vein walls to allow blood to flow from the pulmonary vein(s) directly into various right-side chamber(s) and/or vessel(s) of the heart.

In some implementations, the present disclosure relates to a method of shunting blood. The method comprises accessing a left atrium of a heart with a surgical instrument, advancing the surgical instrument into a pulmonary vein fluidly coupled to the left atrium, forming an opening in a wall portion of the pulmonary vein, and shunting blood from the pulmonary vein into a right-side structure of the heart through the opening in the wall portion of the pulmonary vein.

The wall portion can be a common wall between the pulmonary vein and the right-side structure.

In some embodiments, the right-side structure is a superior vena cava. In some embodiments, the right-side structure is a right atrium of the heart. In some embodiments, the right-side structure is a pulmonary artery.

In some embodiments, the pulmonary vein is a right superior pulmonary vein. In some embodiments, the pulmonary vein is a right inferior pulmonary vein.

The method may further comprise deploying a shunt structure within the opening to retain the opening in the wall of the pulmonary vein. For example, the method may further comprise anchoring the shunt structure to an interior wall of the right-side structure.

In some embodiments, accessing the left atrium is achieved via a transcatheter access path and the surgical instrument comprises a catheter.

The method may further comprise forming an opening in a wall portion of the right-side structure, wherein said shunting blood is through the opening in the wall portion of the right-side structure. For example, the method further comprises securing the opening in the wall portion of the pulmonary vein to the opening in the wall portion of the right-side structure.

In some implementations, the present disclosure relates to a method of shunting blood. The method comprises accessing an interior of a left-side structure of a heart with a surgical instrument, forming an opening in a wall portion of a superior vena cava of the heart, and shunting blood from the left-side structure to the superior vena cava via the opening in the wall portion of the superior vena cava.

The left-side structure may be a pulmonary vein. For example, the pulmonary vein may be a right superior pulmonary vein.

In some embodiments, the wall portion of the superior vena cava is a common wall between the superior vena cava and the left-side structure.

The method can further comprise deploying a shunt structure within the opening to retain the opening.

In some implementations, the present disclosure relates to a method of shunting blood. The method comprises accessing a left atrium of a heart with a tissue-cutting means, advancing the tissue-cutting means into a pulmonary vein fluidly coupled to the left atrium, forming an opening in a wall portion of the pulmonary vein using the tissue cutting means, and shunting blood from the pulmonary vein into a right-side structure of the heart through the opening in the wall portion of the pulmonary vein.

In some embodiments, the tissue-cutting means comprises at least one blade. In some embodiments, the tissue-cutting means comprises a tissue ablation transducer. In some embodiments, the tissue-cutting means comprises a needle.

The method can further comprise dilating the opening using a dilator device.

The method can further comprise deploying a channel-retention means in the opening. For example, the channel-retention means can comprise a wireform stent.

For purposes of summarizing the disclosure, certain aspects, advantages and novel features have been described. It is to be understood that not necessarily all such advantages may be achieved in accordance with any particular embodiment. Thus, the disclosed embodiments may be carried out in a manner that achieves or optimizes one advantage or group of advantages as taught herein without necessarily achieving other advantages as may be taught or suggested herein.

### BRIEF DESCRIPTION OF THE DRAWINGS

Various embodiments are depicted in the accompanying drawings for illustrative purposes and should in no way be interpreted as limiting the scope of the inventions. In addition, various features of different disclosed embodiments can be combined to form additional embodiments, which are part of this disclosure. Throughout the drawings, reference numbers may be reused to indicate correspondence between reference elements.

FIG. 1 is an anterior view of an example human heart.

FIG. 2 is a posterior view of an example human heart.

FIG. 3 is a cross-sectional view of an example human heart.

FIG. 4 is a superior cross-sectional view of the heart of FIG. 3.

FIG. 5A is a cross-sectional view of a heart having a pulmonary vein shunt opening in accordance with one or more embodiments.

FIG. 5B is a superior cross-sectional view of the heart of Figure SA having a pulmonary vein shunt opening in accordance with one or more embodiments.

FIG. 6 is a superior cross-sectional view of a heart having a pulmonary vein shunt implant device implanted therein in accordance with one or more embodiments.

FIG. 7 is a superior cross-sectional view of a heart having a pulmonary vein shunt implant device implanted therein in accordance with one or more embodiments.

FIG. 8 is a superior cross-sectional view of a heart having a grommet-type pulmonary vein shunt implant device implanted therein in accordance with one or more embodiments.

FIG. 9 illustrates an embodiment of a stent-type shunt implant device in accordance with one or more embodiments.

FIG. 10 illustrates an embodiment of a grommet-type shunt implant device in accordance with one or more embodiments.

FIG. 11 is a cutaway view of a human heart and associated vasculature showing certain catheter access paths for pulmonary vein shunting procedures in accordance with one or more embodiments.

FIG. 12 is a flow diagram illustrating a process for forming a pulmonary vein shunt in accordance with one or more embodiments.

FIG. 13 provides cross-sectional images of cardiac anatomy and certain devices corresponding to the process of FIG. 12 according to one or more embodiments.

FIGS. 14-1 and 14-2 show posterior views of hearts having pulmonary vein shunts formed in right inferior pulmonary veins in accordance with one or more embodiments.

FIG. 15 shows a posterior view of a heart having a shunt formed in a right superior pulmonary vein and a right pulmonary artery in accordance with one or more embodiments.

FIG. 16 shows a posterior view of a heart having a shunt formed in a left superior pulmonary vein and a left pulmonary artery in accordance with one or more embodiments.

### DETAILED DESCRIPTION

The headings provided herein are for convenience only and do not necessarily affect the scope or meaning of the claimed invention.

Although certain preferred embodiments and examples are disclosed below, it should be understood that the inventive subject matter extends beyond the specifically disclosed embodiments to other alternative embodiments and/or uses and to modifications and equivalents thereof. Thus, the scope of the claims that may arise herefrom is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments; however, the order of description should not be construed to imply that these operations are order dependent. Additionally, the structures, systems, and/or devices described herein may be embodied as integrated components or as separate components. For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

Certain standard anatomical terms of location are used herein to refer to the anatomy of animals, and namely humans, with respect to various embodiments. Although certain spatially relative terms, such as “outer,” “inner,” “upper,” “lower,” “below,” “above,” “vertical,” “horizontal,” “top,” “bottom,” and similar terms, are used herein to describe a spatial relationship of one device/element or anatomical structure to another device/element or anatomical structure, it is understood that these terms are used herein for ease of description to describe the positional relationship between element(s)/structures(s), as illustrated in the drawings. It should be understood that spatially relative terms are intended to encompass different orientations of the element(s)/structures(s), in use or operation, in addition to the orientations depicted in the drawings. For example, an element/structure described as “above” another element/structure may represent a position that is below or beside such other element/structure with respect to alternate orientations of the subject patient or element/structure, and vice-versa. It should be understood that spatially relative terms, including those listed above, may be understood relative to a respective illustrated orientation of a referenced figure.

### Cardiac Anatomy

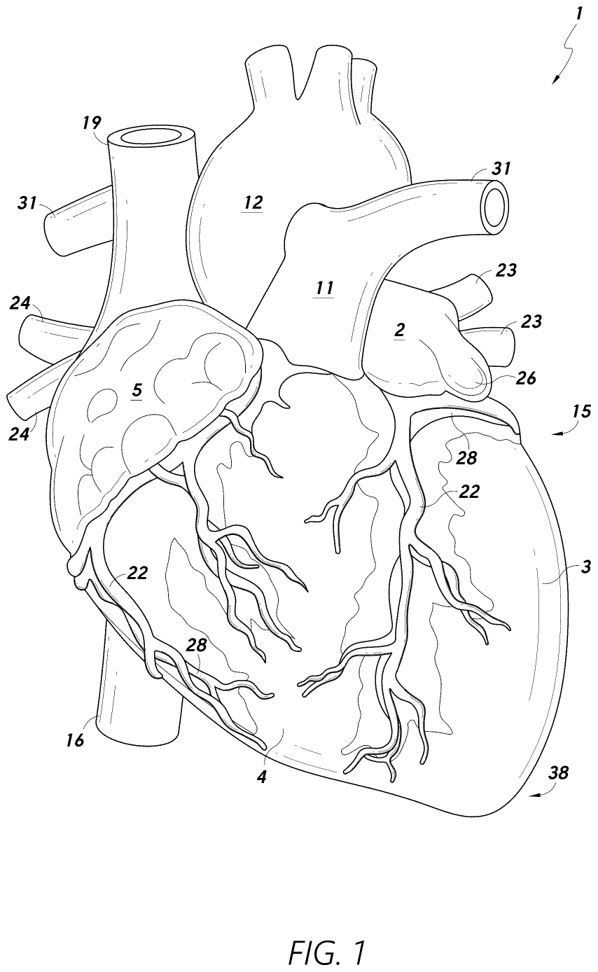

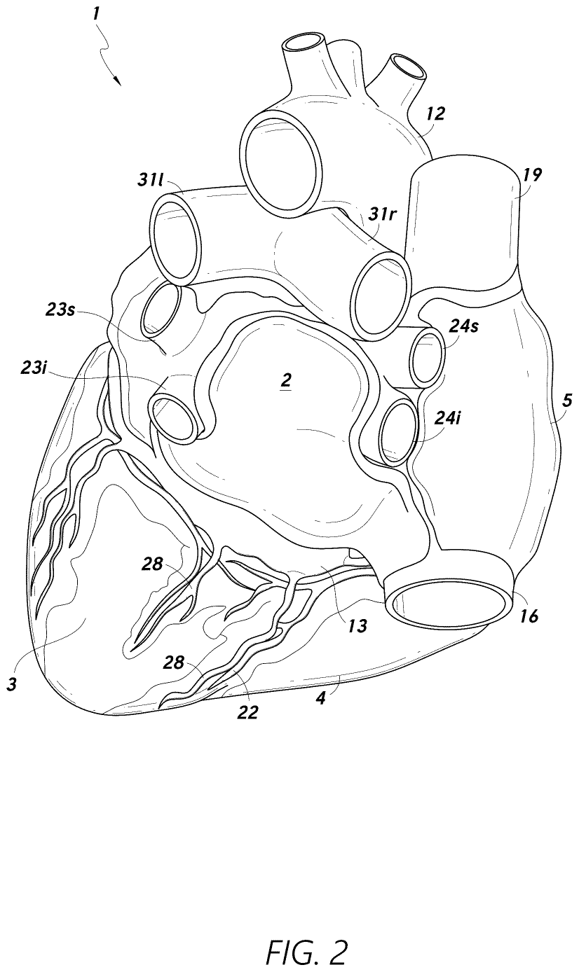

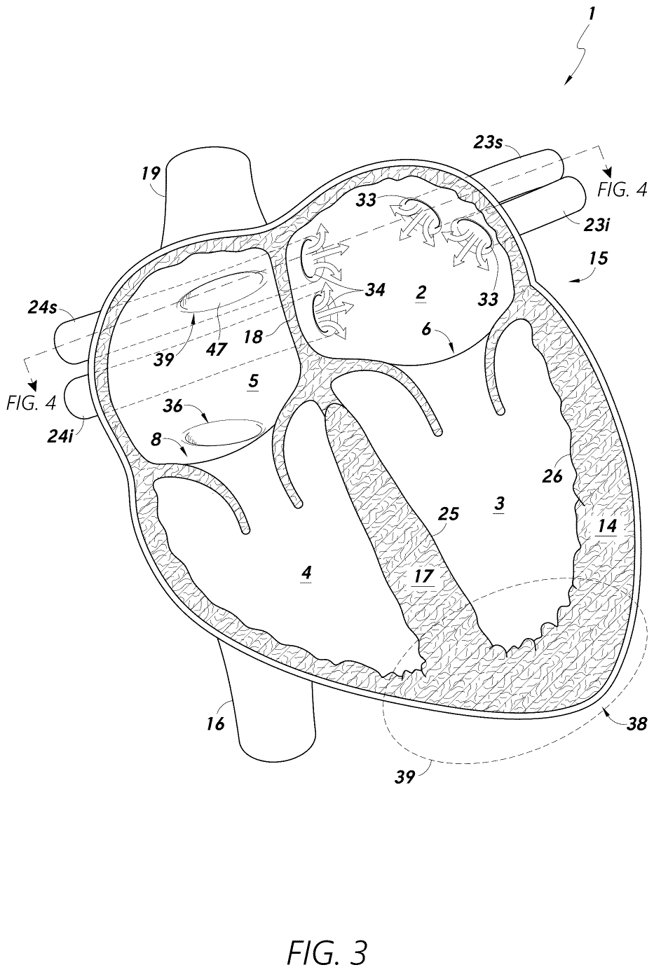

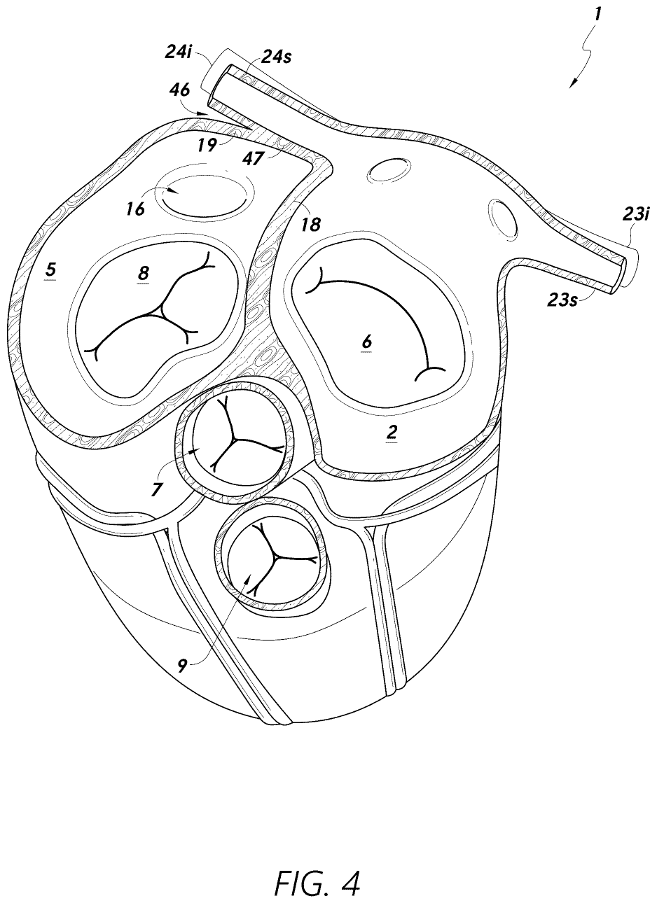

The present disclosure relates to systems, devices, and methods for shunting blood from a pulmonary vein to a right-side chamber/vessel of a heart. Therefore, aspects of the present disclosure relate to the anatomy of the heart, and so the description below includes description of cardiac anatomy for the purpose of providing context for the various inventive features presented herein. FIG. 1 is an anterior view of an example human heart 1; human anatomy is presented and discussed for convenience, although the inventive features presented herein are applicable to any mammalian cardiac anatomy. FIG. 2 is a posterior view of the heart 1, whereas FIG. 3 is a frontal cross-sectional view of the heart 1 and FIG. 4 is a superior cross-sectional view of the heart 1.

With reference to FIGS. 1-4, the heart 1 includes four chambers, namely the left ventricle 3, the left atrium 2, the right ventricle 4, and the right atrium 5. A wall of muscle, referred to as the septum, separates the left-side chambers from the right-side chambers. In particular, an atrial septum wall portion 18 (referred to herein as the “atrial septum,” “interatrial septum,” or “septum”) separates the left atrium 2 from the right atrium 5, whereas a ventricular septum wall portion 17 (referred to herein as the “ventricular septum,” “interventricular septum,” or “septum”) separates the left ventricle 3 from the right ventricle 4. The inferior tip 38 of the heart 1 is referred to as the apex and is generally located on or near the midclavicular line, in the fifth intercostal space.

The heart 1 further includes four valves for aiding the circulation of blood therein. Heart valves may generally comprise a relatively dense fibrous ring, referred to herein as the annulus, as well as a plurality of leaflets or cusps attached to the annulus. Generally, the size and position of the leaflets or cusps may be such that when the heart contracts, the resulting increased blood pressure produced within the corresponding heart chamber forces the leaflets at least partially open to allow flow from the heart chamber. As the pressure in the heart chamber subsides, the pressure in the subsequent chamber or blood vessel may become dominant and press back against the leaflets. As a result, the leaflets/cusps come in apposition to each other, thereby closing the flow passage.

The valves of the heart 1 include the tricuspid valve 8, which separates the right atrium 5 from the right ventricle 4. The tricuspid valve 8 may generally have three cusps or leaflets and may generally close during ventricular contraction (i.e., systole) and open during ventricular expansion (i.e., diastole). The valves of the heart 1 further include the pulmonary valve 9, which separates the right ventricle 4 from the pulmonary artery 11 and may be configured to open during systole so that blood may be pumped toward the lungs, and close during diastole to prevent blood from leaking back into the heart from the pulmonary artery 11. The pulmonary valve 9 generally has three cusps/leaflets, wherein each one may have a crescent-type shape. The heart 1 further includes the mitral valve 6, which generally has two cusps/leaflets and separates the left atrium 2 from the left ventricle 3. The mitral valve 6 may generally be configured to open during diastole so that blood in the left atrium 2 can flow into the left ventricle 3, and advantageously closes during diastole to prevent blood from leaking back into the left atrium 2. Lastly, the aortic valve (not shown in FIGS. 1 and 2; see FIG. 9) separates the left ventricle 3 from the aorta 12. The aortic valve 7 is configured to open during systole to allow blood leaving the left ventricle 3 to enter the aorta 12, and close during diastole to prevent blood from leaking back into the left ventricle 3.

The atrioventricular (i.e., mitral and tricuspid) heart valves are generally associated with a sub-valvular apparatus (not shown), including a collection of chordae tendineae and papillary muscles securing the leaflets of the respective valves to promote and/or facilitate proper coaptation of the valve leaflets and prevent prolapse thereof. The papillary muscles, for example, may generally comprise finger-like projections from the ventricle walls. The chordae tendineae generally keep the valve leaflets from opening in the wrong direction, thereby preventing blood to flow back to the left atrium 2.

Surrounding the ventricles (3, 4) are a number of arteries 22 that supply oxygenated blood to the heart muscle and a number of veins 28 that return the blood from the heart muscle to the right atrium 5 via the coronary sinus 13, which is a relatively large vein that extends generally around the upper portion of the left ventricle 3 and provides a return conduit for blood returning to the right atrium 5. The pulmonary artery 11 carries deoxygenated blood from the right side of the heart to the lungs. The pulmonary artery includes a pulmonary trunk 11 and left 31ₗ and right 31ᵣ pulmonary arteries that branch off of the pulmonary trunk 11, as shown.

The primary roles of the chambers of the left side of the heart (i.e., left atrium 2 and left ventricle 3) are to act as holding chambers for blood returning from the lungs (not shown) and to act as a pump to transport blood to other areas of the heart. The left atrium 2 receives oxygenated blood from the lungs via the pulmonary veins 23, 24. The oxygenated blood that is collected from the pulmonary veins 23, 24 in the left atrium 2 enters the left ventricle 3 through the mitral valve 6. In some patients, the walls of the left atrium 2 are slightly thicker than the walls of the right atrium 5. Deoxygenated blood enters the right atrium 5 through the inferior 16 and superior 19 venae cavae. The right side (i.e., right atrium 5 and right ventricle 4) of the heart then pumps this deoxygenated blood into the pulmonary arteries 31 around the lungs. There, fresh oxygen enters the blood stream, and the blood moves to the left side of the heart via the network of pulmonary veins 23, 24 that ultimately terminate at the left atrium 2, as shown.

The ostia 33, 34 of the pulmonary veins are generally located at or near the posterior left atrial wall of the left atrium 2. The right pulmonary veins 24 carry blood from the right lung (not shown) to the left atrium 2, where it is distributed to the rest of the circulatory system as described in detail herein. The right pulmonary veins 24 include the right inferior pulmonary vein 24ᵢ and the right superior pulmonary vein 24ₛ, as shown. Meanwhile, the left pulmonary veins 23 generally include the left inferior pulmonary vein 23ᵢ and the left superior pulmonary vein 23ₛ. The left pulmonary veins 23 generally carry blood from the left lung (now shown) into the left atrium 2, where it continues to flow to the rest the body.

The left ventricle 3 is the primary pumping chamber of the heart 1. A healthy left ventricle is generally conical or apical in shape in that it is longer (with respect to the mean electrical axis of the heart) than it is wide (with respect to a transverse axis extending between opposing walls of the left ventricle at their widest point) and descends from a base 15 with a decreasing cross-sectional diameter and/or circumference to the point or apex 14. Generally, the apical region of the heart can be considered the bottom region of the heart that is within the left and/or right ventricular region but is distal to the mitral 6 and tricuspid 8 valves and disposed toward the tip 38 of the heart.

The pumping of blood from the left ventricle 3 is accomplished by a squeezing motion and a twisting or torsional motion. The squeezing motion occurs between the lateral walls of the left ventricle 3 and the septum 17. The twisting motion is a result of contraction of heart muscle fibers that extend in a generally circular or spiral direction around the heart. When these fibers contract, they produce a gradient of angular displacements of the myocardium from the apex 14 to the base 15 about the mean electrical axis of the heart. The resultant force vectors extend at angles from about 30-60 degrees to the flow of blood through the aortic valve and ascending aorta 12. The contraction of the heart 1 is manifested as a counterclockwise rotation of the apex 14 relative to the base 15, when viewed from the apex 14 (i.e., inferior view of the heart 1). The contractions of the heart 1, in connection with the filling volumes of the left atrium 2 and ventricle 3, respectively, can result in relatively high fluid pressures in the left side of the heart at least during certain phase(s) of the cardiac cycle, the results of which are discussed in detail below. Attached to the left atrium 2 is the left atrial appendage 26, which generally may comprise a muscular ear-shaped pouch. The left atrial appendage 26 (also referred to as the left auricle) is thought to function as a decompression chamber during left ventricular systole and during other periods when left atrial pressure is high.

### Elevated Left-Side Cardiac Pressures

Fluid volume and pressure conditions associated with the various cardiac chambers and anatomy described above can impact the health of a patient. For example, congestive heart failure is a condition associated with the relatively slow movement of blood through the heart and/or body, which can cause the fluid pressure in one or more chambers of the heart to increase, particularly in the left side of the heart. For example, when the left ventricle fails or when the mitral valve fails, left atrial pressure can increase substantially. As a result, the heart may not pump sufficient oxygen to meet the body's needs. Increased left atrial volume and pressure can further result in abnormal P waves in cardiac electrical signals.

The various chambers of the heart may respond to pressure increases by stretching to hold more blood to pump through the body or by becoming relatively stiff and/or thickened. The walls of the heart can eventually weaken and become unable to pump as efficiently. In some cases, the kidneys may respond to cardiac inefficiency by causing the body to retain fluid. Fluid build-up in arms, legs, ankles, feet, lungs, and/or other organs can cause the body to become congested, which is referred to as congestive heart failure. Generally, left atrial pressure may be relatively highly correlated with risk of congestive heart failure. Furthermore, there may generally be a relatively strong correlation between increases in left atrial pressure and pulmonary congestion. Acute decompensated congestive heart failure is a leading cause of morbidity and mortality, and therefore treatment and/or prevention of congestive heart failure is a significant concern in medical care. Embodiments of the present disclosure can serve to treat and/or prevent congestive heart failure through reduction in left atrial pressure in patients suffering from high left atrial pressure. Generally, increases in ventricular filling pressures associated with diastolic and/or systolic heart failure can occur prior to the occurrence of symptoms that lead to hospitalization. For example, cardiac pressure indicators may present weeks prior to hospitalization in some patients. Therefore, reduction in left atrial and/or ventricular pressure in accordance with embodiments of the present disclosure may advantageously be implemented as a preventative measure to reduce risks of hospitalization and/or the onset of heart failure.

Determination of high left atrial pressure may be made in any suitable or desirable way. Dyspnea represents a cardiac pressure indicator characterized by shortness of breath or the feeling that one cannot breathe well enough. Dyspnea may result from elevated atrial pressure, which may cause fluid buildup in the lungs from pressure back-up. Therefore, it may be desirable to implement certain left atrial pressure reduction solutions presented herein in response to dyspnea symptoms. Additionally or alternatively, left atrial pressure reduction in accordance with embodiments or the present disclosure may be implemented prior to manifestation of dyspnea symptoms and/or other symptoms/complications through direct and/or indirect pressure (e.g., left atrial pressure) monitoring and/or intervention. For example, left atrial pressure monitoring may be implemented using one or more sensors implanted or disposed in one or more chambers of the heart, such as within the left atrium. In some implementations, left atrial pressure may be derived or inferred through measurement of other chambers or vessels of the heart, which may serve as surrogates of left atrial pressure. For example, measurements of pressure in one or more of the right atrium, right ventricle, pulmonary artery, and/or pulmonary artery wedge may indicate elevated left atrial pressure.

### Pulmonary Vein Shunting

In some implementations, the present disclosure relates to systems, devices, and methods for reducing fluid volume and/or pressure in the left side of the heart, such as in the left atrium, left ventricle, and/or pulmonary veins, through the shunting of blood through wall(s) of one or more of the pulmonary veins. For example, shunting in accordance with aspects of the present disclosure can involve forming a shunt flow path opening between a pulmonary vein, such as the right superior pulmonary vein, and the superior vena cava and/or right atrium.

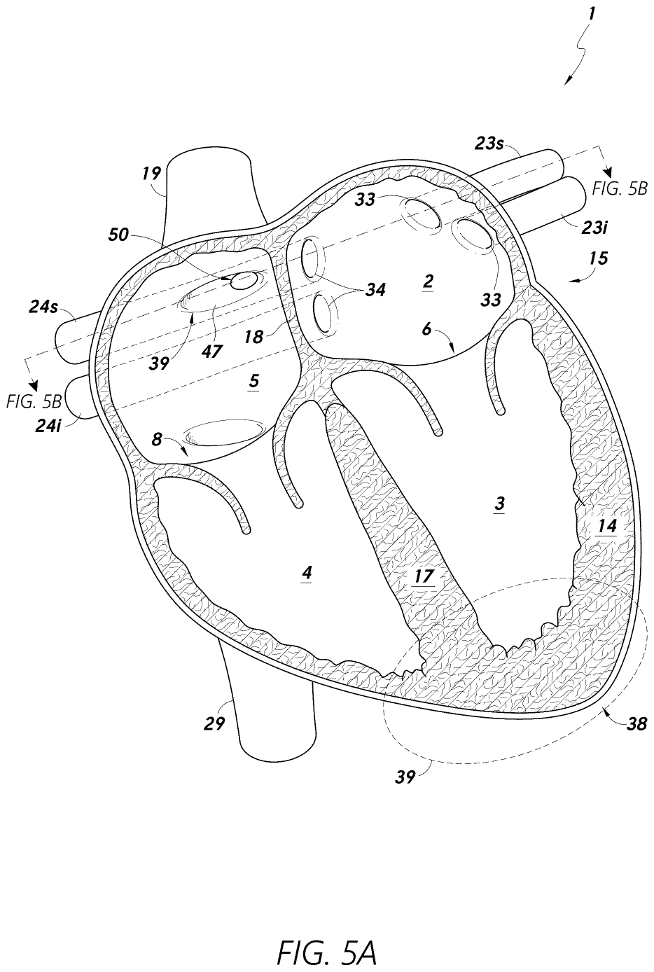

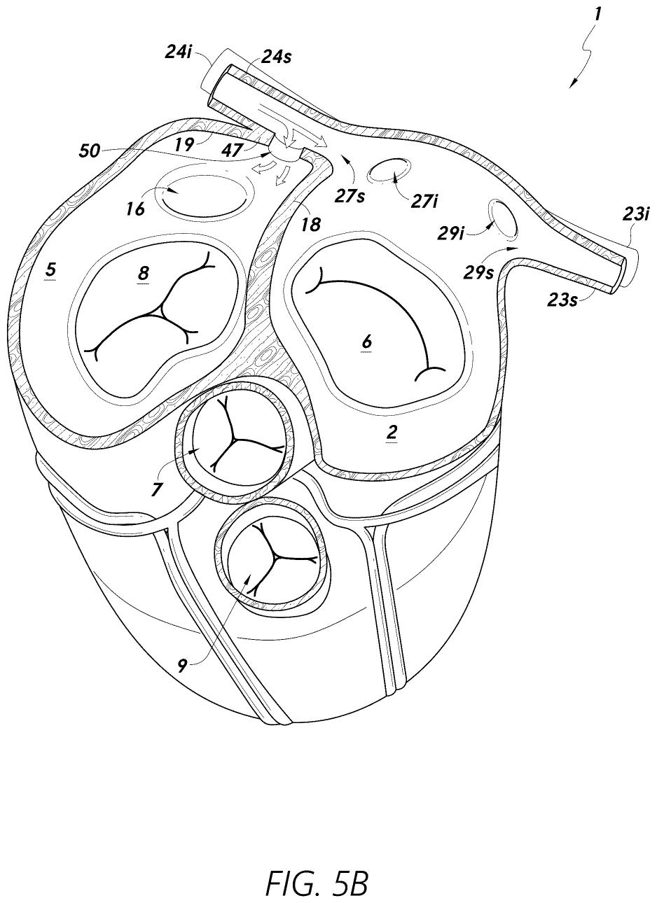

FIGS. 5A and 5B show frontal and superior cross-sectional views, respectively, of a heart 1 having a pulmonary vein shunt opening 50 in accordance with one or more embodiments of the present disclosure. In some patients, the anatomy of the right superior pulmonary vein 24ₛ is positioned and oriented such that a common wall 47 between the pulmonary vein 24ₛ and the superior vena cava 19 may be present, such as at or near an area of the superior vena cava that opens into the right atrium 5. Therefore, an opening 50 formed in the common wall 47 may allow for the shunting of blood from the pulmonary vein 24 into the right atrium 5 without leakage of blood outside of the heart.

The common wall portion 47 may be present near the septal boundary between the right atrium 5 and the left atrium 2. For example, the ostium 27ₛ of the right superior pulmonary vein 24ₛ may likewise be positioned in an area that is relatively close to the interatrial septum 18, wherein an area near the opening/ostium 27ₛ within the pulmonary vein 24ₛ on a generally anterior side of the pulmonary vein 24ₛ may be shared with the wall of the superior vena cava 19 and/or right atrium 5.

Although certain embodiments are described herein as including or involving a shunt opening formed in a wall separating the right superior pulmonary vein 24ₛ from the right atrium 5, in some patients, a suitable shared wall area between the pulmonary vein 24ₛ and the right side of the heart may be below the superior vena cava ostium 39, such that shunt opening 50 opens directly into the right atrium 5 rather than into the superior vena cava 19. Therefore, description herein of pulmonary vein shunts, such as shunt openings and shunt implant devices, between a pulmonary vein and the superior vena cava should be understood to also relate to and disclose shunts between the pulmonary vein and the right atrium below the ostium of the superior vena cava.

In some embodiments, the shunt opening 50 is formed by forming an opening 50 in the wall 47, wherein the shunt opening 50 may be a temporary shunt that may be permitted to close-/heal-over over time. That is, the opening 50 may be formed without implantation of a shunt/opening reinforcement structure or device. The shunt opening 50 may be formed using a transcatheter delivery system including one or more tools configured to form an opening in the tissue wall 47. For example, the opening 50 may be formed using an ablation tool, such as ultrasonic and/or radio-frequency radiation ablation tool or similar. Such ablation tool may be configured to burn the tissue of the wall 47 to cause the opening 50 to be formed therein through the application of ultrasonic energy and/or radiofrequency radiation, or the like, emitted using an energy transducer component. For example, cardiac tissue ablation may be implemented by delivering energy, such as ultrasound or radiofrequency electromagnetic radiation energy, through a catheter or other transducer device to the target area of the pulmonary vein wall. Such energy can ablate or destroy relatively small focal areas of the cardiac tissue and form an opening therein. Ablation may further be implemented to cauterize the tissue around an opening formed in the pulmonary vein wall using other means (e.g., needle/wire, cutting blade, and/or dilator). In addition to ultrasound and radiofrequency radiation ablation, other types of catheter ablation can be implemented. For example, cardiac tissue ablation may be implemented using cryoablation, which generally utilizes a pressurized refrigerant in a catheter tip or other device to ablate the target tissue. According to some solutions, ablation can be implemented using minimally invasive techniques, such as through transcatheter access to the target atrium and/or other area of the heart. Transcatheter shunt formation and/or implantation in connection with embodiments of the present disclosure may be particularly desirable with respect to patients suffering from elevated left-side cardiac pressures, which may generally be considered a high-risk patient demographic may be at risk of injury and/or other complications from surgical interventions.

Additional solutions can be implemented for producing the opening 50 in the wall 47. Such means/mechanism(s) may advantageously be implemented using transcatheter procedures. Example means/tools that may be implemented to form pulmonary vein shunt openings in tissue walls in accordance with aspects of the present disclosure may include one or more blades, needles, wires, and/or other devices having a relatively sharp point or edge and configured to be penetrated/cut through a cardiac tissue wall. In some implementations, a cutting tool configured to excise a cut-out portion of the tissue wall and remove the cut-out portion of tissue using the delivery system may be used to form the pulmonary vein shunt opening.

The implementation of a shunt from within a pulmonary vein may provide certain advantages or benefits relative to certain other shunt positions/locations. For example, the position within the pulmonary vein may provide a position relatively far away from the mitral valve 6, such that any structure or implant that may be utilized in connection with the shunting may be relatively far from the mitral valve and less inclined to obstruct flow within the left atrium. Furthermore, the size of the pulmonary vein 24 may be of sufficient diameter to allow for catheter access for the purpose of forming the shunt opening 50 and/or deploying an implant device therein. Furthermore, by shunting blood directly into the superior vena cava and or right atrium, such blood flow may not result in any undesirable altering of fluid dynamics in any chamber or blood vessel of the right side of the heart. For example, where blood is shunted into a blood vessel that is smaller than the superior vena cava and right atrium, such shunted blood flow may result in undesirable pressurization and/or the forcing of blood upstream, possibly resulting in undesirable pooling, dilation, and/or other adverse physiological effects. With particular respect to shunting into the superior vena cava, where the patient is in a generally upright orientation, gravity may serve to direct the shunted fluid down into the right atrium, resulting in relatively insignificant impact on the fluid dynamics of the right side of the heart.

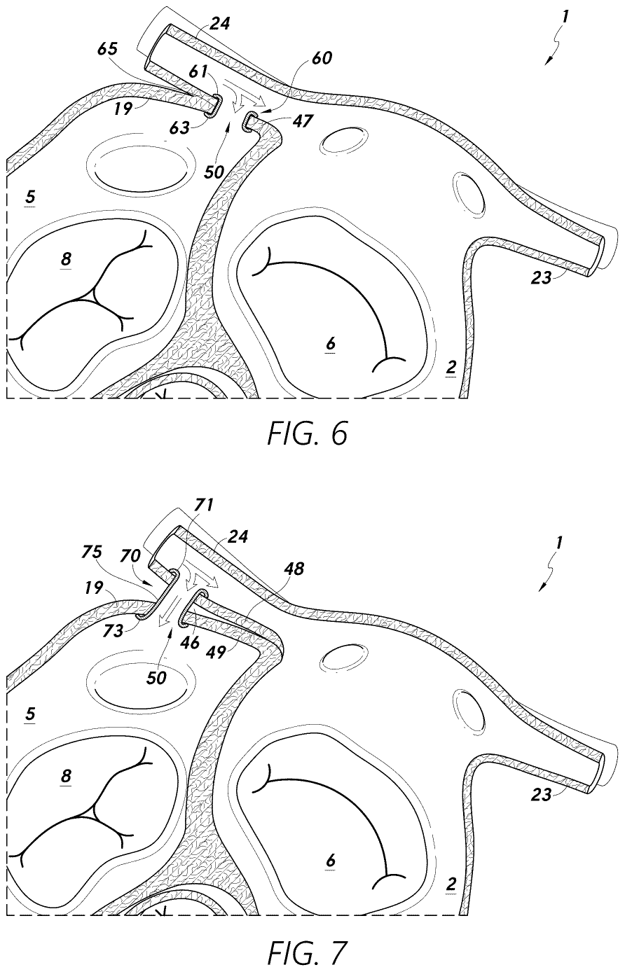

FIG. 6 is a superior cross-sectional view of a heart 1 having a pulmonary vein shunt implant device 60 implanted therein in accordance with one or more embodiments of the present disclosure. As described in detail above, embodiments of the present disclosure relate to the formation of shunt pathways from within pulmonary veins to provide shunting from the left side of the heart the right side of the heart. In some implementations, embodiments of the present disclosure further involve the implantation of certain shunt structures configured to retain a shunt opening through a pulmonary vein wall postoperatively. For example, FIG. 6 shows a tubular or other-shaped conduit structure 60 used as a shunt implant device deployed within a shunt opening 50 to provide retention support therefore. It should be understood that any of the embodiments disclosed herein may be implemented using a shunt implant structure to hold open the shunt opening and/or define a desired shunt flow path. Furthermore, it should be understood that shunt implant devices in accordance with aspects of the present disclosure may have any suitable or desirable form or shape. The shunt implant device 60 may be implanted to provide a shunt from a superior or inferior right pulmonary vein 24, or from either of the left-side pulmonary veins 23.

The shunt implant device 60 may be implanted in the opening 50 in the common wall 47 between the pulmonary vein 24 and the superior vena cava 19 and/or right atrium 5. In some embodiments, the shunt implant device 60 includes a conduit portion 65 that defines the shunt flow pathway. The conduit portion 65 may have any suitable or desirable cross-sectional shape or area, such as circular, oblong, ovoid, elliptical, rectangular, or other shape. In some embodiments, the shunt implant device 60 is anchored to one or more of the pulmonary vein 24 and the superior vena cava 19 or right atrium 5. For example, the shut implant device 60 may include one or more proximal pulmonary vein anchors 61 configured to secure the conduit device 60 to the interior wall of the pulmonary vein 24. Additionally or alternatively, the shunt implant device 60 may include one or more distal anchors 63, which may be configured to secure the conduit device to the interior wall of the superior vena cava 19 and/or right atrium 5. In some embodiments, the conduit portion 65 of the shunt implant device 60 is at least partially rigid, such that the form thereof may be substantially maintained over time after implantation.

FIG. 7 is a superior cross-sectional view of a heart 1 having a pulmonary vein shunt implant device 70 implanted therein in accordance with one or more embodiments. Certain embodiments are disclosed herein relating to shunt openings and implants disposed/formed in a common wall between a pulmonary vein and a right-side blood vessel or chamber (e.g., superior vena cava, right atrium). However, in some patients, sufficient area of common wall between such vessel/chambers may not be present and/or it may be desirable to form a pulmonary vein shunt in an area that is not a common wall between the pulmonary vein and the target right-side vessel/chamber. Therefore, in some embodiments, a shunt implant device 70 may be implanted in a manner as to bridge between a pulmonary vein 24 and a right-side vessel/chamber, wherein the walls associated with such vessel/chambers in the area of the shunt implant device are not common, but rather separate walls that contact one another and/or are separated by a gap 46 in one or more areas. For example, as shown in FIG. 7, the right-side pulmonary vein 24 (e.g., right superior pulmonary vein) may have a wall 48 on a side of the pulmonary vein oriented towards the target right-side vessel/chamber, wherein an opposing wall 49 of the right-side vessel/chamber (e.g., superior vena cava 19 and/or right atrium 5) is separate from the pulmonary vein wall 48 in the area of the shunt opening 50. In at least some areas, a gap 46 may be present between the opposing walls.

When a pulmonary vein shunt is formed in an area where there is no common wall between the pulmonary vein and the target right-side vessel/chamber, it may be necessary or desirable for a conduit structure 70 to be implanted in order to prevent blood leakage outside of the heart into the chest cavity through the shunt opening. For example, the shunt implant device 70 may advantageously be fluid-tight at least in the conduit portion 75 thereof to ensure that blood shunted from the pulmonary vein 24 passes into the target right-side vessel/chamber. In some embodiments, the proximal 71 and/or distal 73 anchor features of the shunt implant device 70 may include certain flange features designed to prevent leakage of blood around the anchors. For example, such/features may be secured to the respective interior cardiac walls in a manner as to prevent blood from leaking under the anchors/flanges and out around the outside of the conduit portion 75. Exemplary features of shunt implant devices as shown in FIGS. 6 and 7 are described in greater detail below with respect to FIG. 9.

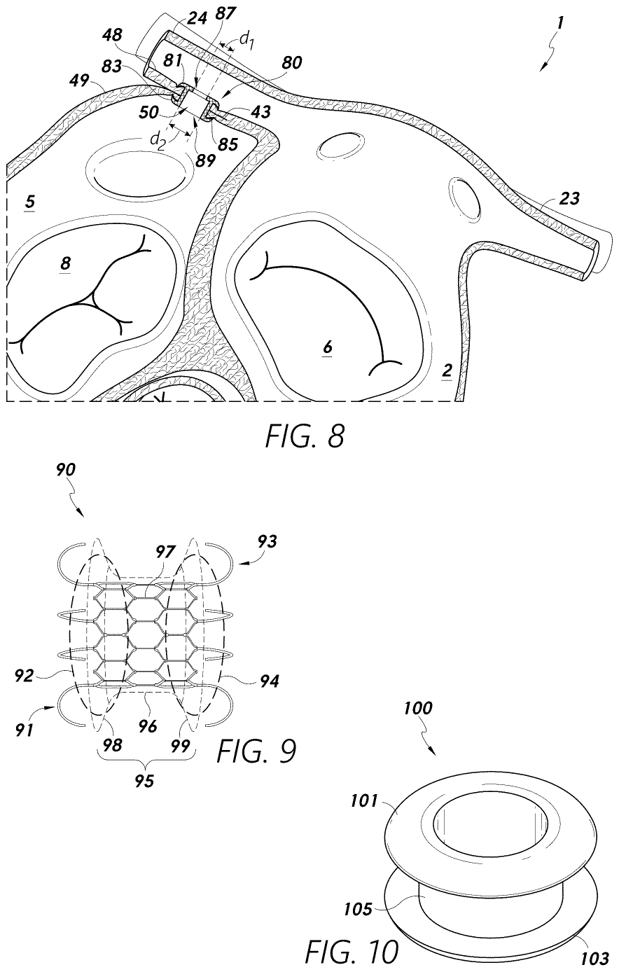

FIG. 8 is a superior cross-sectional view of a heart 1 having a grommet-type pulmonary vein shunt implant device 80 implanted therein in accordance with one or more embodiments of the present disclosure. As described above, in areas where a pulmonary vein shunt path is desired, such area may not be entirely aligned with a common wall area between the pulmonary vein and the target right-side vessel/chamber. Therefore, as described above in connection with FIG. 7, it may be desirable to implant a shunt implant device that spans any gap/distance between the source pulmonary vein and the target right-side vessel/chamber. In some cases, it may be desirable to implant a shunt implant device that allows for the source pulmonary vein and the target right-side vessel/chamber to be clamped together to shorten the distance between such vessels/chambers and/or to provide a secure/safe shunt pathway, as shown in FIG. 8. Such shunt implant devices (e.g., grommet-type shunt structure 80) can be at least partially rigid and include distal and proximal anchors 81, 83 configured to secure the conduit portion 85 in place and/or clamp the blood vessel walls together.

As described, the grommet-style shunt implant device 80 may serve to clamp separate vessel/chamber walls together in order to provide a fluid-tight seal between the blood vessels/chambers and/or to secure the implant device 80 in place when shunting through separate vessel/chamber walls. Furthermore, grommet-style shunt implant devices as shown in FIG. 8 may be utilized in shunting through common walls between a pulmonary vein and right-side chamber/vessel. For example, the anchor flanges 81, 83 may serve to prevent or reduce tearing or abrasion of the tissue wall(s) around a formed shunt opening. That is, regardless of whether a shunt pathway/conduit spans a single common wall or two separate walls, the structure of the conduit portion 85 and the anchor flanges 81, 83 may maintain the integrity of the tissue wall around the opening 50 to prevent undesired tearing or other damage that may result in hemorrhage or other injury to the patient.

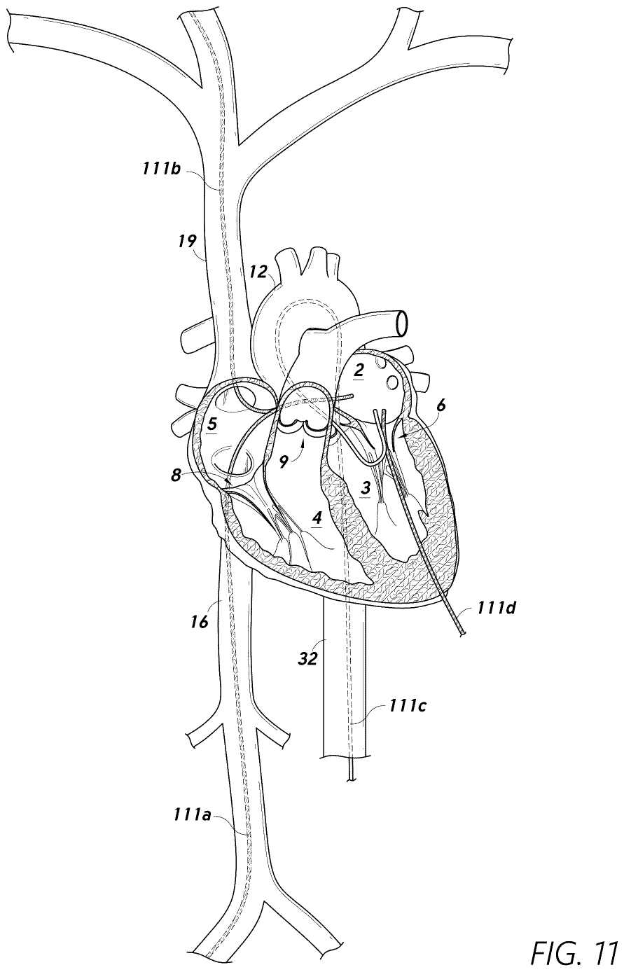

In some embodiments, a grommet-style shunt implant device as shown in FIG. 8 may include a proximal/inlet opening 87 and a distal/outlet opening 88, wherein the diameters and/or flow areas associated therewith may be different with respect to the proximal/inlet opening 87 and the distal/outlet opening 88. For example, in some embodiments, the proximal opening 87 may have a diameter d₁ that is designed to control/produce a desired amount of shunt flow, wherein the dimension d₁ of the proximal/inlet opening 87 is different (e.g., smaller/narrower) than the distal/outlet opening 88. In some embodiments, by utilizing a narrower inlet opening 87 compared to the outlet opening 88, backflow from the right side of the heart into the pulmonary vein 24 may be inhibited due to the relatively higher pressure at the inlet opening 87 compared to the outlet opening 88. Further details relating to grommet-style shunt implant devices are described below in connection with FIG. 10.

FIG. 9 illustrates an embodiment of a stent-type shunt implant device 90 in accordance with one or more embodiments of the present disclosure. The shunt implant device 90 may be an expandable or non-expandable implant device, and represents an example shunt implant device that may be utilized in connection with any of the disclosed embodiments. In some embodiments, the shunt implant device 90 comprises a self-expanding stent or frame component 97, which may be shaped and configured to form a conduit, such as a cylindrical-cross-sectional conduit, or conduit having any other suitable or desirable cross-sectional shape. Although certain self-expanding frames and conduits are described herein as examples in some contexts, in some embodiments, a shunt implant device may be balloon-expandable or may not require expansion after deployment (e.g., from a delivery system). In some embodiments, the shunt implant device 90 is not collapsible and/or expandable. For example, the implant device 90 may be implanted in a substantially deployed/expanded configuration in connection with a surgical or transcatheter (e.g., transfemoral) procedure or access to the heart of a patient.

Opposite ends 92, 94 of the conduit portion 95 of the device 90 (e.g., as formed by the frame 97) can be coupled to and/or otherwise associated with one or more anchor components 91, 93, respectively. The image of FIG. 9 shows barb-/hook-type tissue anchors 91, 93. However, it should be understood that any types of anchors may be utilized in connection with shunt implant devices in accordance with embodiments of the present disclosure. In addition, or as an alternative, to the barb-/hook-type anchors 91, 93, the ends of the shunt implant device 90 may have flange or other-type anchor features 98, 99, which may be configured to hold the device 90 in position within a shunt opening when the features 98, 99 have a diameter or other dimension that is greater than the diameter or other dimension of the shunt opening, as described herein. The flange-type features 98, 99 can be fully circumferential or may be disposed around only a portion of the circumference or outer perimeter of the shunt device 90. In some implementations, the flange features 98, 99 are configured to be disposed within a chamber/vessel of a heart and help prevent withdrawal therefrom of the end(s) of the device 90. For example, the anchor features 98, 99 can advantageously project radially from the conduit axis such that they have a dimension (e.g., width, diameter, etc.) that is greater than a width/diameter of the conduit portion 95 of the device 90.

The tissue anchors 91, 93 shown in FIG. 9, as well as those described in connection with other embodiments of the present disclosure, may be any suitable or desirable types of tissue anchors. For example, in some embodiments, a tissue anchor associated with a shunt implant device comprises a pre-shaped wireform, such as a loop, coil, spiral, or the like, which may be configured to assume a relatively wide tissue anchor profile once deployed. Other types of tissue anchors that may be used include, but are not limited to, tension-fit or resistance-fit tissue anchors, such as stents or the like, barb-type tissue anchors, which may incorporate tip features configured to resist withdrawal of the anchor tip(s) from tissue in which it is embedded following embedding, corkscrew-type tissue anchors, and/or other types of tissue anchors that may or may not be known in the art.

As described in detail herein, the shunt implant device 90 may advantageously be dimensioned to have a length sufficient to traverse the distance from the target inflow implantation site (e.g., an interior of a pulmonary vein) to the target outflow implantation site (e.g., superior vena cava, right atrium). In some embodiments, the shunt implant device 90 advantageously includes a covering 96, which may be disposed within or without the frame 97, and may be at least partially fluid-tight to thereby facilitate funneling or channeling of blood flow through the medial conduit portion 95 of the shunt implant device 90. The shunt implant device 90 may comprise a covered stent or graft structure. For example, the device 90 may be covered with thin PTFE material, or other material or biological tissue. The anchor(s) 91, 93 of the inflow and outflow ends may be anchored/implanted in the relevant biological tissue in any suitable or desirable way.

FIG. 10 illustrates an embodiment of a grommet-type shunt implant device 100 in accordance with one or more embodiments of the present disclosure. As described above, the grommet-type shunt implant device 100 may be configured to clamp/secure the wall of a pulmonary vein and provide a shunt conduit therethrough. For example, the shunt implant device 100 may be used to clamp/hold the wall of a pulmonary vein to the wall of a superior vena cava and/or right atrium, wherein such walls may be a common wall or may be separate wall segments (e.g., separated by a gap and/or in contact with one another).

The implant device 100 may be configured to be inserted into a shunt opening in the pulmonary vein tissue wall, thereby at least partially filling the opening with the conduit portion 105 of the implant device. The implant device 100 further includes one or more flared or collared flange features 101, 103 configured to secure the implant device to one or more blood vessel/chamber walls, as described herein. In some embodiments, the flange features 101, 103 may be configured to be extended/projected axially with respect to an axis of the conduit 105 for delivery/transport thereof. That is, with the flange features actually projected, the grommet-type shunt implant device 100 may have a generally cylindrical form, wherein upon deployment, the flange features 101, 103 may be configured to flare radially outward to provide clamping/securing surfaces to hold the implant device 100 in place. For example, the flared flange features 101, 103 may comprise shape-memory metal configured to assume the flared configuration shown in FIG. 10 upon deployment from a delivery catheter or other delivery device/system. In some embodiments, when deployed, the flared features 101, 103, are angled at least partially towards one another to clamp or pinch the tissue wall(s) to which they are secured.

### Pulmonary Vein Shunting Processes

According to some solutions, pulmonary vein shunting can be achieved using a transcatheter approach, wherein a catheter/tool may be delivered to the left atrium, and ultimately the target pulmonary vein, through the vasculature of the patient, such as through one or more arteries or veins of the arm, groin, or neck. FIG. 11 is a cutaway view of a human heart and associated vasculature showing certain catheter access paths for pulmonary vein shunting procedures in accordance with one or more embodiments.

FIG. 11 shows various catheters 111 that may be used to implement pulmonary vein shunting in accordance with aspects of the present disclosure. The catheters 111 can advantageously be steerable and relatively small in cross-sectional profile to allow for traversal of the various blood vessels and chambers through which they may be advanced en route to, for example, the left atrium 2 or other anatomy or chamber. Catheter access to the left atrium 2 in accordance with certain transcatheter pulmonary vein shunting solutions may be made by first accessing the right atrium 5 via the inferior vena cava 16 (as shown by the catheter 111a) or the superior vena cava 19 (as shown by the catheter 111b) and crossing the atrial septum (e.g., in the area at or near the fossa ovalis) to access the left atrium 2.

Although access to the left atrium is illustrated and described in connection with certain examples as being via the right atrium and/or vena cavae, such as through a transfemoral or other transcatheter procedure, other access paths/methods may be implemented in accordance with examples of the present disclosure. For example, in cases in which septal crossing through the interatrial septal wall is not possible, other access routes may be taken to the left atrium 2. In patients suffering from a weakened and/or damaged interatrial septum, further engagement with the septal wall can be undesirable and result in further damage to the patient. Furthermore, in some patients, the septal wall may be occupied with one or more implant devices or other treatments, wherein it is not tenable to traverse the septal wall in view of such treatment(s). As alternatives to transseptal access, transaortic access may be implemented, wherein a delivery catheter 111c is passed through the descending aorta 32, aortic arch 12, ascending aorta, and aortic valve 7, and into the left atrium 2 through the mitral valve 6. Alternatively, transapical access may be implemented to access the target anatomy, as shown by delivery catheter 111d.

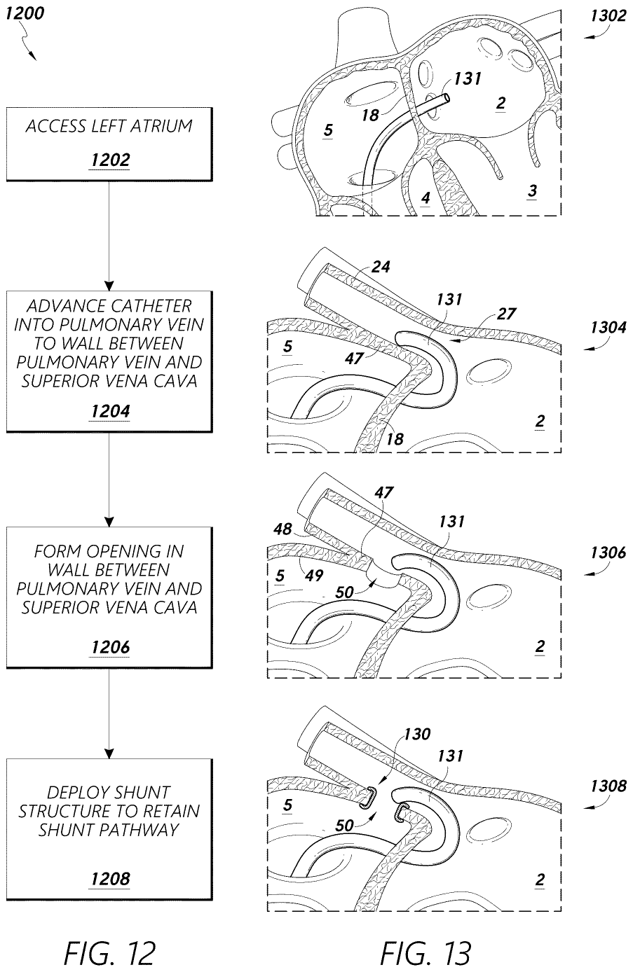

FIG. 12 is a flow diagram illustrating a process 1200 for forming a pulmonary vein shunt in accordance with one or more embodiments. FIG. 13 provides cross-sectional images of cardiac anatomy and certain devices corresponding to the process 1200 of FIG. 12 according to one or more embodiments of the present disclosure.

At block 1202, the process 1200 involves accessing the left atrium 2 of the heart of a patient. For example, access may be made via a transcatheter approach as described in detail herein. In some implementations, a delivery catheter 131 may be advanced to the right atrium 5 and further advanced through the interatrial septum 18 to access the left atrium 2, as shown in image 1302.

At block 1204, the process 1200 involves advancing the catheter 131 into a target pulmonary vein 24, such as a right superior pulmonary vein, as described in detail herein. The process 1200 may advantageously involve advancing the distal end of the catheter 131 to a wall portion 47 between the pulmonary vein 24 and the superior vena cava and/or right atrium 5. For example, the wall portion 47 may be a common wall between the pulmonary vein 24 and the superior vena cava and/or right atrium 5. Although shown as a common wall 47, it should be understood that the wall portion targeted for shunt formation may be a portion of a pulmonary vein wall 48 that is separate from the superior vena cava and/or right atrium wall 49.

At block 1206, the process 1200 involves forming an opening 50 in the pulmonary vein wall 47. For example, as stated above, the wall 47 may be a common wall between the pulmonary vein 24 and the superior vena cava and/or right atrium. The opening 50 may be formed in any suitable or desirable way, such as through wire/needle puncture, cutting, ablation, dilation, and or any combination thereof. With the opening 50 formed in the wall(s) 47 (and/or 48, 49), the process 1200 may or may not involve, at block 1208, deploying a shunt structure 130 within the opening 50 to retain the shunt pathway 50 post-implantation.

### Additional Pulmonary Vein Shunting Areas

Various embodiments are described above relating to the formation of shunts between a right superior pulmonary vein and a right-side chamber/vessel of the heart. However, it should be understood that pulmonary vein shunting in accordance with aspects of the present disclosure may involve shunting from other pulmonary veins as well, wherein a shunt pathway may be formed and/or implanted between such other pulmonary vein(s) and a right-side chamber or vessel of the heart.

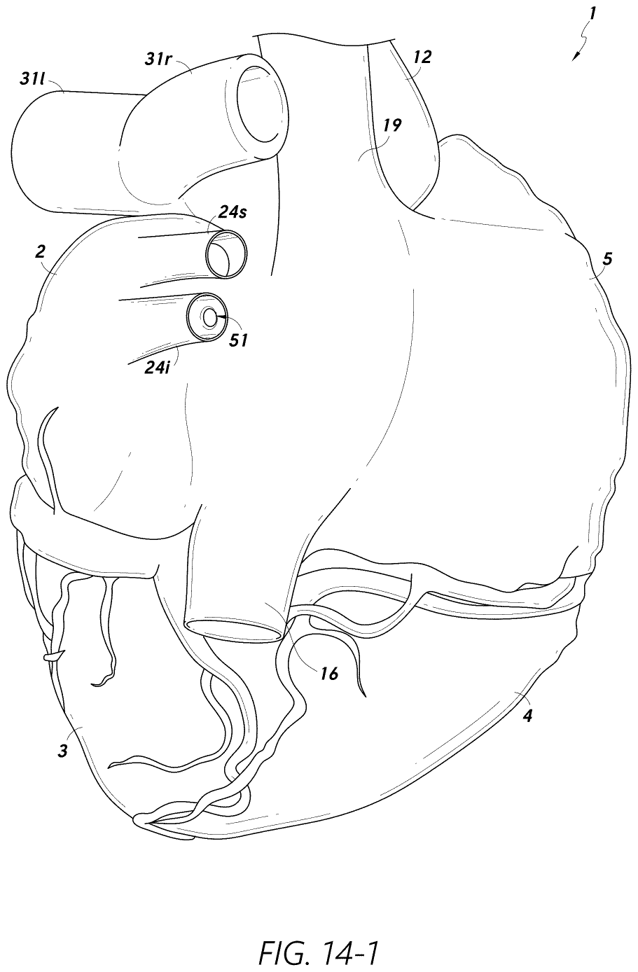

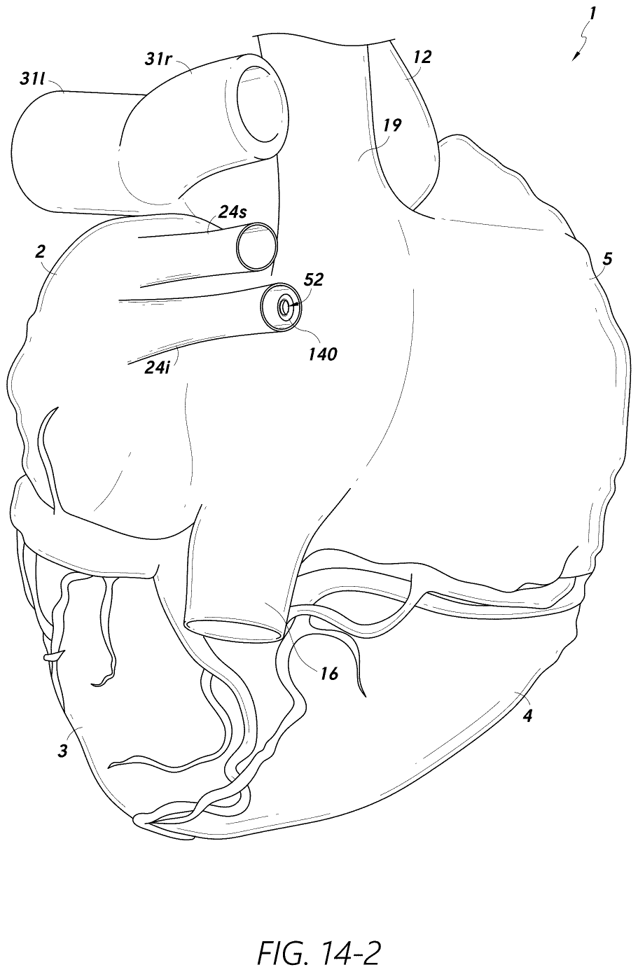

FIGS. 14-1 and 14-2 show posterior views of hearts having pulmonary vein shunts formed in right inferior pulmonary veins in accordance with one or more embodiments of the present disclosure. For example, as with other embodiments disclosed herein, the shunting solution shown in FIG. 14-1 can involve the formation of an opening 51 in a common wall between the right inferior pulmonary vein 24ᵢ and the right atrium 5 and/or one of the vena cavae. The opening 51 shown in FIG. 14-1 may or may not be reinforced with a shunt implant device, such as a grommet- or stent-type shunt implant device, as described in detail herein.

FIG. 14-2 shows a shunt 52 in the right inferior pulmonary vein 24ᵢ providing a shunt into the right atrium 5 in an area where no common wall exists between the pulmonary vein 24ᵢ and the right side of the heart. As shown, a grommet-type shunt implant device 140 may be implanted to clamp the wall of the pulmonary vein 24ᵢ to the wall of the right atrium 5 and/or one of the vena cavae. The grommet implant device 140 can serve to hold the blood vessel/chamber walls together and to form a fluid-tight seal around the opening 52.

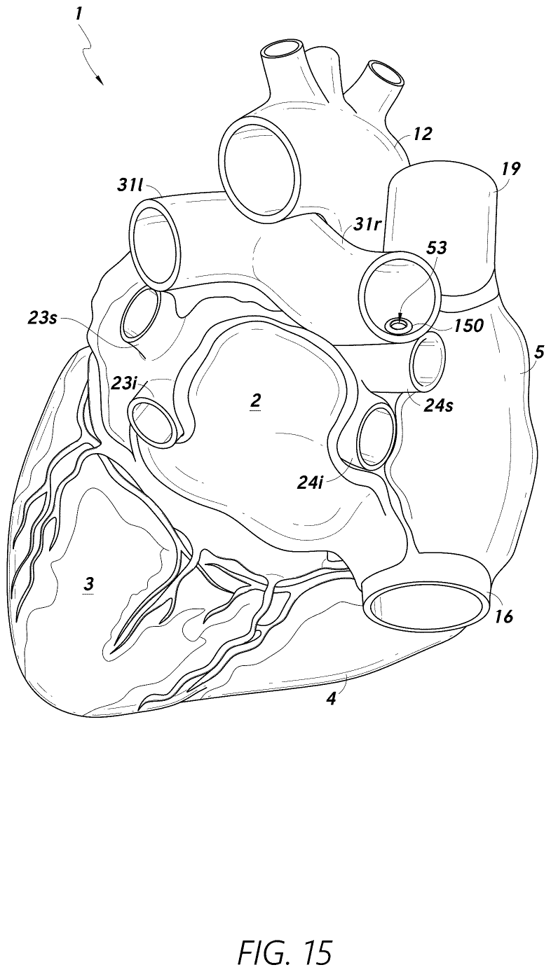

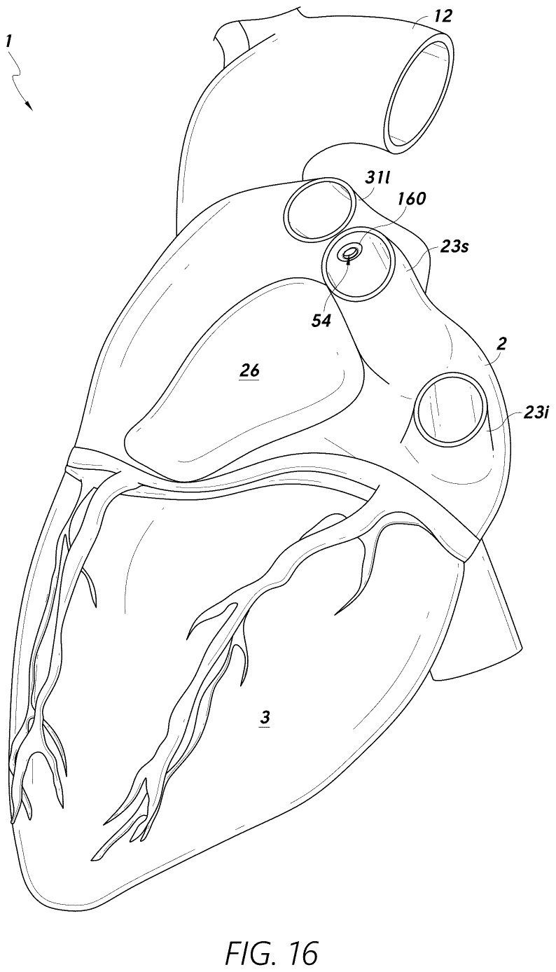

FIG. 15 shows a posterior view of a heart having a shunt 53 formed in a right superior pulmonary vein 23ₛ and right pulmonary artery 31ᵣ in accordance with one or more embodiments. Although a common wall between the pulmonary vein 24ₛ and the pulmonary artery 31ᵣ may generally not be present, embodiments of the present disclosure may involve forming a fluid-tight conduit between such vessels, as shown in FIG. 15. For example, the blood vessels may be clamped together using a shunt implant device 150, such as a grommet-type shunt implant device as described in detail herein. The shunt 53 may allow oxygenated blood in the pulmonary vein 24ₛ to flow into the deoxygenated blood flow in the pulmonary artery 31ᵣ flowing to the right lung (not shown).

FIG. 16 shows a posterior view of a heart having a shunt 54 formed in a left superior pulmonary vein 23ₛ and left pulmonary artery 31/in accordance with one or more embodiments. Although a common wall between the pulmonary vein 23, and the pulmonary artery 31ₗ may generally not be present, embodiments of the present disclosure may involve forming a fluid-tight conduit between such vessels, as shown in FIG. 16. For example, the blood vessels may be clamped together using a shunt implant device 160, such as a grommet-type shunt implant device as described in detail herein. The shunt 54 may allow oxygenated blood in the pulmonary vein 23ₛ to flow into the deoxygenated blood flow in the pulmonary artery 31; flowing to the left lung (not shown).

### ADDITIONAL EMBODIMENTS

Depending on the embodiment, certain acts, events, or functions of any of the processes or algorithms described herein can be performed in a different sequence, may be added, merged, or left out altogether. Thus, in certain embodiments, not all described acts or events are necessary for the practice of the processes.

Conditional language used herein, such as, among others, “can,” “could,” “might,” “may.” “e.g.,” and the like, unless specifically stated otherwise, or otherwise understood within the context as used, is intended in its ordinary sense and is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous, are used in their ordinary sense, and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y and Z,” unless specifically stated otherwise, is understood with the context as used in general to convey that an item, term, element, etc. may be either X, Y or Z. Thus, such conjunctive language is not generally intended to imply that certain embodiments require at least one of X, at least one of Y and at least one of Z to each be present.

It should be appreciated that in the above description of embodiments, various features are sometimes grouped together in a single embodiment, Figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Moreover, any components, features, or steps illustrated and/or described in a particular embodiment herein can be applied to or used with any other embodiment(s). Further, no component, feature, step, or group of components, features, or steps are necessary or indispensable for each embodiment. Thus, it is intended that the scope of the inventions herein disclosed and claimed below should not be limited by the particular embodiments described above, but should be determined only by a fair reading of the claims that follow.

It should be understood that certain ordinal terms (e.g., “first” or “second”) may be provided for ease of reference and do not necessarily imply physical characteristics or ordering. Therefore, as used herein, an ordinal term (e.g., “first,” “second,” “third,” etc.) used to modify an element, such as a structure, a component, an operation, etc., does not necessarily indicate priority or order of the element with respect to any other element, but rather may generally distinguish the element from another element having a similar or identical name (but for use of the ordinal term). In addition, as used herein, indefinite articles (“a” and “an”) may indicate “one or more” rather than “one.” Further, an operation performed “based on” a condition or event may also be performed based on one or more other conditions or events not explicitly recited.

Unless otherwise defined, all terms (including technical and scientific terms) used herein have the same meaning as commonly understood by one of ordinary skill in the art to which example embodiments belong. It be further understood that terms, such as those defined in commonly used dictionaries, should be interpreted as having a meaning that is consistent with their meaning in the context of the relevant art and not be interpreted in an idealized or overly formal sense unless expressly so defined herein.

The spatially relative terms “outer,” “inner,” “upper,” “lower,” “below,” “above,” “vertical,” “horizontal,” and similar terms, may be used herein for ease of description to describe the relations between one element or component and another element or component as illustrated in the drawings. It be understood that the spatially relative terms are intended to encompass different orientations of the device in use or operation, in addition to the orientation depicted in the drawings. For example, in the case where a device shown in the drawing is turned over, the device positioned “below” or “beneath” another device may be placed “above” another device. Accordingly, the illustrative term “below” may include both the lower and upper positions. The device may also be oriented in the other direction, and thus the spatially relative terms may be interpreted differently depending on the orientations.

Unless otherwise expressly stated, comparative and/or quantitative terms, such as “less,” “more,” “greater,” and the like, are intended to encompass the concepts of equality. For example, “less” can mean not only “less” in the strictest mathematical sense, but also, “less than or equal to.”

What is claimed is:

1. A method for reducing cardiac pressure by shunting blood from an oxygenated cardiac blood vessel a deoxygenated cardiac blood vessel, the method comprising:
accessing a left atrium of a heart with a surgical instrument;
advancing the surgical instrument into a left pulmonary vein fluidly coupled to the left atrium;
forming a first opening in a wall portion of the left pulmonary vein;
forming a second opening in a wall portion of a left pulmonary artery;
deploying a shunt implant through the first opening and the second opening, the shunt implant being sized and shaped to extend between the left pulmonary vein and the left pulmonary artery and to define a shunt flow path therebetween; and
shunting blood from the left pulmonary vein into the left pulmonary artery through the shunt flow path.

2. The method of claim 1, wherein the left pulmonary vein is a left superior pulmonary vein.

3. The method of claim 1, wherein the shunt implant comprises a conduit portion extending between the left pulmonary vein and the left pulmonary artery and a plurality of anchoring features disposed at opposite ends of the conduit portion.

4. The method of claim 3, wherein the plurality of anchoring features comprise radially projecting flanges configured to engage opposing walls of the left pulmonary vein and the left pulmonary artery to clamp the walls of the left pulmonary vein and the left pulmonary artery together.

5. The method of claim 3, wherein the plurality of anchoring features comprise hook-shaped elements configured to penetrate tissue and resist axial withdrawal of the shunt implant.

6. The method of claim 1, wherein the shunt implant comprises a frame defining the shunt flow path and a covering disposed on the frame, the covering being at least partially fluid-tight.

7. The method of claim 1, wherein forming at least one of the first opening or the second opening comprises applying ablation energy using an ultrasound transducer or a radiofrequency energy source.

8. The method of claim 7, further comprising cauterizing tissue surrounding at least one of the first opening or the second opening to reduce bleeding and inhibit tissue ingrowth.

9. The method of claim 1, wherein deploying the shunt implant comprises drawing the left pulmonary vein and the left pulmonary artery toward one another to reduce a gap between the left pulmonary vein and the left pulmonary artery prior to anchoring the shunt implant.

10. A method for reducing cardiac pressure by shunting blood from an oxygenated cardiac blood vessel a deoxygenated cardiac blood vessel, the method comprising:
advancing an ablation instrument into a left atrium of a heart via a transcatheter access path;
advancing the ablation instrument from the left atrium into a pulmonary vein fluidly coupled to the left atrium;
using the ablation instrument to deliver ablation energy to cardiac tissue separating the pulmonary vein from a right-side structure of the heart, thereby forming an opening through the cardiac tissue; and
shunting blood from the pulmonary vein into the right-side structure of the heart through the opening.

11. The method of claim 10, wherein the opening is formed and maintained without deploying an implant device to maintain patency of the opening.

12. The method of claim 10, wherein the ablation energy comprises radiofrequency energy.

13. The method of claim 10, wherein the ablation energy comprises ultrasound energy.

14. The method of claim 10, wherein using the ablation instrument further comprises cauterizing tissue surrounding the opening to inhibit bleeding and tissue ingrowth.

15. The method of claim 10, wherein the pulmonary vein is a right superior pulmonary vein and the right-side structure comprises a right atrium or a superior vena cava.

16. The method of claim 10, wherein forming the opening comprises directing the ablation energy against a common wall between the pulmonary vein and the right-side structure.

17. The method of claim 10, wherein the opening is formed in a manner as to permit shunting of blood for a period of time and to subsequently close through natural tissue healing.

18. A method for reducing cardiac pressure by shunting blood from an oxygenated cardiac blood vessel a deoxygenated cardiac blood vessel, the method comprising:
accessing a left atrium of a heart with a surgical instrument;
advancing the surgical instrument into a pulmonary vein fluidly coupled to the left atrium;
forming a first opening in a wall portion of the pulmonary vein;
forming a second opening in a wall portion of a pulmonary artery;
deploying a shunt implant through the first opening and the second opening, the shunt implant being sized and shaped to extend between the pulmonary vein and the pulmonary artery and to define a shunt flow path therebetween; and
shunting blood from the pulmonary vein into the pulmonary artery through the shunt flow path.

19. The method of claim 18, wherein the pulmonary vein is a right pulmonary vein and the pulmonary artery is a right pulmonary artery.

20. The method of claim 18, further comprising drawing the pulmonary vein and the pulmonary artery against one another prior to deploying the shunt implant, wherein deploying the shunt implant involves clamping tissue of the pulmonary vein and tissue of the pulmonary artery together with the shunt implant to establish a fluid-tight shunt pathway.
